(19) United States
(12) Patent Application Publication
(10) Pub. No.: US 2007/0286890 A1
(43) Pub. Date: Dec. 13, 2007

(54) Eyelash applicator and method

(76) Inventor: John Garnett Walt, Huntington Beach, CA (US)

Correspondence Address: WALTER A. HACKLER, Ph.D.;PATENT LAW OFFICE, SUITE B, 2372 S.E. BRISTOL STREET, NEWPORT BEACH, CA, 92660-0755, US

(21) Appl. No.: 11/449,432

(22) Filed: Jun. 7, 2006

(51) Int. Cl. A61K 31/557; A61K 9/70

(52) U.S. Cl. 424/443; 514/573

(57) ABSTRACT

A hair growth applicator system and method includes a squeezable container, an application device, and a cyclopentane heptanoic acid, 2-cycloalkyl or arylalkyl compound represented by the formula:

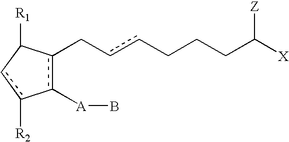

wherein the dashed bonds represent a single or double bond which can be in the cis or trans configuration, A, B, Z, X, R₁ and R₂ are as defined in the specification.

The present invention is generally related to an eye fluid applicator and is more particularly directed to a method for applying a hair growth stimulation formulation and a hair growth application system.

Applicators for the delivery of fluids to an eye have long been used for a great number of purposes. As an example, the common eye bath for comfort, refreshment, or rinsing may include a cup having an anatomically adapted design for emersion of the eye. In addition, delivery of a large fluid amounts by a spray device have also been used for eyewashes in emergency situations.

The present invention is particularly directed to applicators suitable for the administration of very small amounts of fluids to an eyelid by self-administration. In general a medical formulation should be delivered in a well defined volume to assure a specific dose to be delivered and/or absorbed. A large surplus is not desirable due to the possibility of absorbance in non-targeted tissues. This is particularly important for the application of a hair growth stimulation formulation such as set forth in U.S. patent application Ser. No. 10/345,738 filed Jan. 15, 2003 entitled “Method of Enhancing Hair Growth”. This application is to be incorporated herein in its entirety.

Minute volumes are also important with regard to price considerations for expensive medication which require frequent daily administration.

These consideration place severe demands on an applicator for administering a growth stimulation formulation for eyelashes. Small formulation amounts must be positioned with great care in order to prevent the application by overflow to non-targeted areas.

It is further and desirable that administration be conducted in various body positions and independent of applicator orientation.

In addition it is important to maintain sterility of formulation when utilized in an applicator suitable for multiple dosing.

The present invention provides for a method of applying a hair growth stimulation formulations to mammalian skin, particularly an eyelid utilizing a system suitable for multiple dosing yet enabling sterile storage of the formulation.

### SUMMARY OF THE INVENTION

A hair growth application system in accordance with the present invention generally includes a squeezable container, an applicator device, and a formulation disposed in the container, said formulation comprising a cyclopentane heptanoic acid, 2-cycloalkyl or arylalkyl compound represented by the formula I:

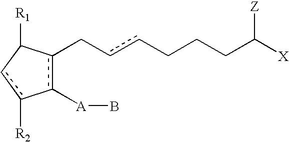

wherein dashed bonds represent a single or double bond which can be in a cis or trans configuration, A is an alkylene or alkenylene radical having from two to six carbon atoms, which radical may be interrupted by one or more oxa radicals and substituted with one or more hydroxy, oxo, alkyloxy or alkylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms,

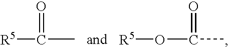

wherein R⁵ is a lower alkyl radical having from one to six carbon atoms; Z is ═O; one of R₁ and R₂ is ═O, —OH or a —O(CO)R₆ group, and the other one is —OH or —O(CO)R₆, or R₁ is ═O and R₂ is H, wherein R₆ is a saturated or unsaturated acyclic hydrocarbon group having from 1 to about 20 carbon atoms, or —(CH₂)mR₇ wherein m is 0 or an integer of from 1 to 10, and R₇ is cycloalkyl radical, having from three to seven carbon atoms, or a hydrocarbyl aryl or heteroaryl radical, as defined above or a pharmacologically acceptable acid addition salt thereof.

Preferably, the concentration of the compound applied is from about 0.0000001% to about 50% by weight of the composition and a maximum of 3 ml of said formulation is disposed in said container.

Preferably, the compound is a cyclopentane heptanoic acid, 2-(phenyl alkyl or phenyloxyalkyl) represented by the formula II:

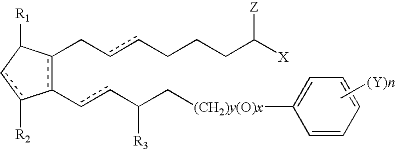

wherein y is 0 or 1, x is 0 or 1 and x and y are not both 1, Y is a radical selected from the group consisting of alkyl, halo, e.g. fluoro, chloro, etc., nitro, amino, thiol, hydroxy, alkyloxy, alkylcarboxy, halo substituted alkyl wherein said alkyl radical comprises from one to six carbon atoms, etc. and n is 0 or an integer of from 1 to 3 and R₃ is ═O, —OH or —O(CO)R₆ wherein R₆ is as defined above or a pharmaceutically acceptable salt thereof.

More preferably the compound is a compound of formula III.

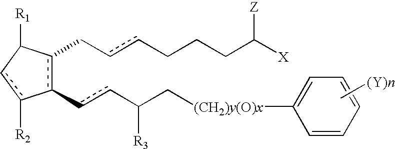

wherein hatched lines indicate α configuration, solid triangles are used to indicate β configuration.

More preferably y is 1 and x is 0 and R₁, R₂ and R₃ are hydroxy.

Most preferably the compound is cyclopentane N-ethyl heptanamide-5-cis-2-(3α-hydroxy-5-phenyl-1-trans-pentenyl)-3,5-dihydroxy, [1₆₀,2_{β},3_{α},5_{α}], also known as bimatoprost.

The applicator device may be an applicator brush, porous foam swab or pad, hollow tube, eye dropper, dip stick, or combination thereof.

The applicator device may be a brush and the system further comprises a passageway interconnecting said brush and the container and the said brush is sized for application of formulation to an eyelid. In addition, a one-way valve disposed in one of the container and the passageway may be provided for preventing reverse flow into the container in order to maintain sterility of the compound in the container. Alternatively, the formulation may be applied to a brush surface in a drop wise maneuver immediately prior to using the brush to apply the formulation to an eyelid. In this manner, close control of the quantity of formulation applied may be effective.

A method of applying a hair growth stimulation formulation in accordance with the present invention includes applying to mammalian skin, in particular an eyelid, a volume of said formulation with an applicator device; said formulation comprising a cyclopentane heptanoic acid, 2-cycloalkyl or arylalkyl compound represented by the formula:

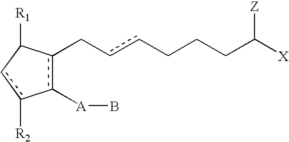

wherein dashed bonds represent a single or double bond which can be in a cis or trans configuration, A is an alkylene or alkenylene radical having from two to six carbon atoms, which radical may be interrupted by one or more oxa radicals and substituted with one or more hydroxy, oxo, alkyloxy or alkylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms,

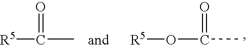

wherein R⁵ is a lower alkyl radical having from one to six carbon atoms; Z is ═O; one of R₁ and R₂ is ═O, —OH or a —O(CO)R₆ group, and the other one is —OH or —O(CO)R₆, or R₁ is ═O and R₂ is H, wherein R₆ is a saturated or unsaturated acyclic hydrocarbon group having from 1 to about 20 carbon atoms, or —(CH₂)mR₇ wherein m is 0 or an integer of from 1 to 10, and R₇ is cycloalkyl radical, having from three to seven carbon atoms, or a hydrocarbyl aryl or heteroaryl radical, as defined above or a pharmacologically acceptable acid addition salt thereof; said formulation being packaged in a container preventing contamination of the formulation.

More particularly, the method of the present invention may include a compound of a formula:

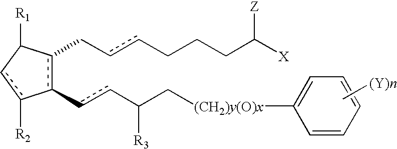

wherein y is 0 or 1, x is 0 or 1 and x and y are not both 1, Y is a radical selected from the group consisting of alkyl, halo, nitro, amino, thiol, hydroxy, alkyloxy, alkylcarboxy, halo substituted alkyl wherein said alkyl radical comprises from one to six carbon atoms, n is 0 or an integer of from 1 to about 3 and R₃ is ═O, —OH or —O(CO)R₆ wherein R₆, hatched lines indicate α configuration and solid triangles are used to indicate β configuration.

### BRIEF DESCRIPTION OF THE DRAWINGS

The advantages and features of the present invention will be better understood by the following description when considered in conjunction with the accompanying drawings, in which:

FIGS. 1 and 2 illustrate the method in accordance with the present invention utilizing a hair growth application system which includes a squeezable container and an applicator device;

FIG. 3 is an exploded view of the hair growth application system in accordance with the present invention in partial cross section illustrating a squeezable container and a breathable, or vented, cap for covering the applicator;

FIG. 4 is a cross sectional view of the applicator, container and cap in an assembled configuration;

FIGS. 5 and 6 illustrates an alternative embodiment of the present invention; and

FIGS. 7A-7D, 8A-8D, 9A-9D, 10A-10D, and 11A-11D show the results of the method and system in accordance with the present invention.

### DETAILED DESCRIPTION

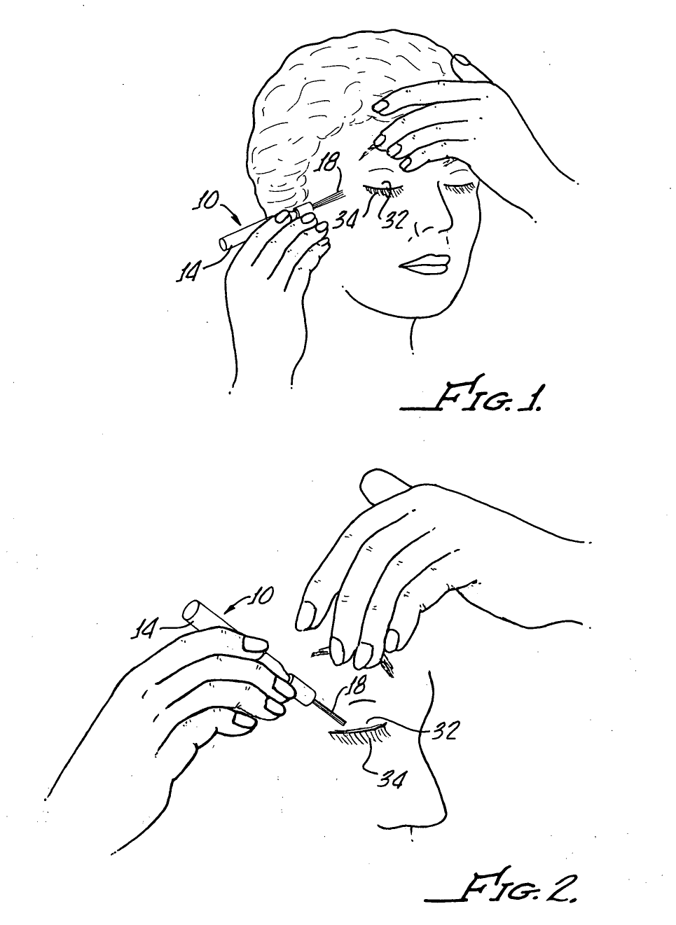

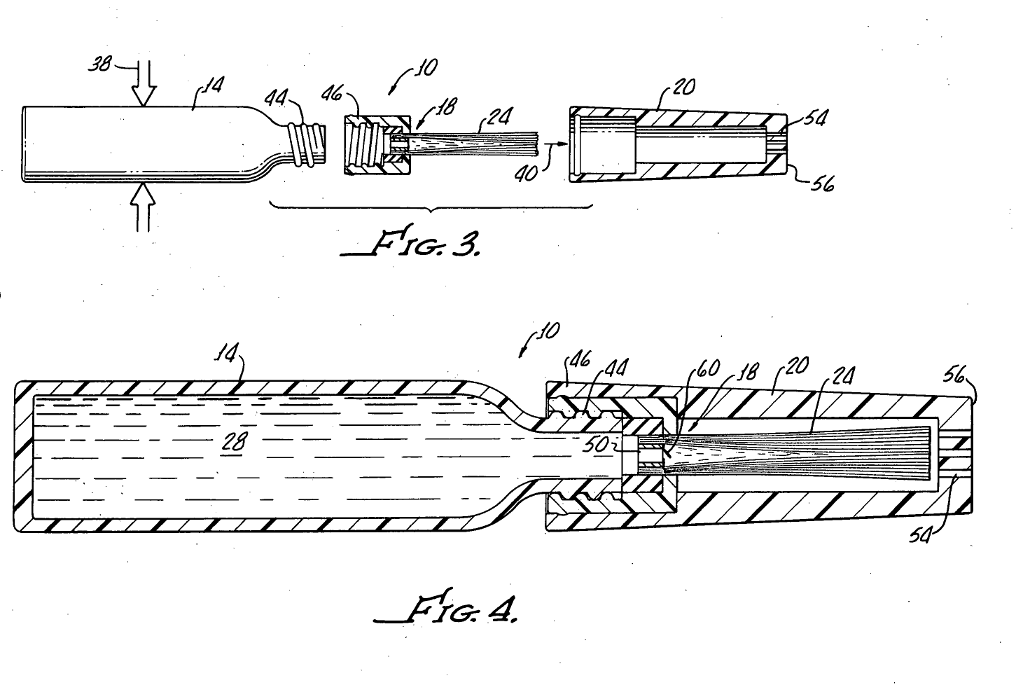

With reference to the FIGS. 1-4, there is shown the hair growth application system 10 which generally includes a squeezable container 14, an applicator device 18, and a cap 20. The Applicator device 18 may include an applicator brush 24 or alternatively, but not shown, a porous foam swab or pad, hollow tube, eye dropper, dipstick, or any combination thereof, not shown for the sake of brevity. The brush 24 is sized for application of a formulation 28 disposed within the container 14 to an eyelid 32 for promoting the growth and thickening of eyelashes 34 extending therefrom, see FIGS. 1 and 2.

As part of the present invention, the formulation 28, disposed in the container 14, is a cyclopentane heptanoic acid, 2-cycloalkyl or arylalkyl compound represented by the formula I:

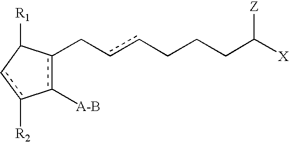

wherein dashed bonds represent a single or double bond which can be in the cis or trans configuration, A is an alkylene or alkenylene radical having from two to six carbon atoms, which radical may be interrupted by one or more oxa radicals and substituted with one or more hydroxy, oxo, alkyloxy or alkylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms,

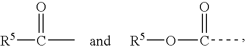

wherein R⁵ is a lower alkyl radical having from one to six carbon atoms; Z is ═O; one of R₁ and R₂ is ═O, —OH or a —O(CO)₆ group, and the other one is —OH or —O(CO)₆, or R₁ is ═O and R₂ is H, wherein R₆ is a saturated or unsaturated acyclic hydrocarbon group having from 1 to about 20 carbon atoms, or —(CH₂)mR₇ wherein m is 0 or an integer of from 1 to 10, and R₇ is cycloalkyl radical, having from three to seven carbon atoms, or a hydrocarbyl aryl or heteroaryl radical, as defined above or a pharmacologically acceptable acid addition salt thereof. A pharmaceutical carrier adapter for topical application to mammalian skin may be provided.

Preferably, the compound is a cyclopentane heptanoic acid, 2-(phenyl alkyl or phenyloxyalkyl) presented by the formula II:

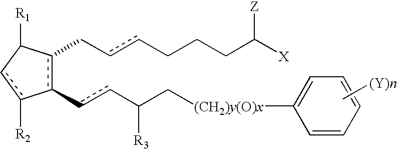

Wherein y is 0 or 1, x is 0 or 1 and x and y are not both 1, Y is a radical selected from the group consisting of alkyl, halo, e.g. fluoro, chloro, etc., nitro, amino, thiol, hydroxy, alkyloxy, alkylcarboxy, halo substituted alkyl wherein said alkyl radical comprises from one to six carbon atoms, etc. and n is 0 or an integer of from 1 to 3 and R₃ is ═O, —OH or —O(CO)R₆ wherein R₆ is as defined above or a pharmaceutically acceptable salt thereof.

More Preferably the compound is a compound of formula III:

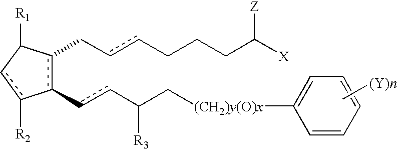

wherein y is 0 or 1, x is 0 or 1 and x and y are not both 1, Y is a radical selected from the group consisting of alkyl, halo, nitro, amino, thiol, hydroxy, alkyloxy, alkylcarboxy, halo substituted alkyl wherein said alkyl radical comprises from one to six carbon atoms, n is 0 or an integer of from 1 to about 3 and R₃ is ═O, —OH or —O(CO)R₆ wherein R₆, hatched lines indicate α configuration and solid triangles are used to indicate β configuration.

More preferably y is 1 and x is 0 and R₁, R₂ and R₃ are hydroxy.

Most preferably the compound is cyclopentane N-ethyl heptanamide-5-cis-2-(3α-hydroxy-5-phenyl-1trans-pentenyl)-3,5-dihydroxy, [1_{α},2_{β},3_{α},5_{α}], also known as bimatoprost (Lumigan®).

A maximum of up to about 3 ml of the formulation 28 is disposed in the squeezable container 14, which may be formed from any suitable material to maintain sterility of the formulation. The container 14 size is selected for enabling convenient handling by a user. As indicated in FIG. 3 by arrows 38, pressure applied to the container 14 squeezes formulation through the bristles 24 as indicated by the arrow 40 in FIG. 3 to effect application of formulation 28 to the eyelid 32, eyelash 34 as shown in FIG. 2. In addition, the concentration of a compound may be from about 0.0000001% to about 50% of the composition.

The applicator device 18, including brush, may be fitted to the container 14 through a threaded neck 44 by a correspondingly threaded fitting 46 supporting the brush in an extended fashion which also provides a passageway 50 interconnecting the brush 24 and the container 14.

The compound 28 being water soluble enables residue free drying of the brush 24 after each application. This is facilitated by vents, or openings, 54 in a cap end 56. Additional vents (not shown) may be provided in the cap 20 to facilitate drying of the brush 24 after use.

In order to maintain the sterility of the formulation 28, a one-way valve 60 may be provided in the passageway 50. Alternatively, although not shown, a valve may be disposed in the container 14 or the threaded neck 44.

An alternative hair growth application system embodiment 70 is shown in FIGS. 5 and 6. In this instance, the system 70 includes a squeezable container 72 along with an applicator device 74 with the hereinabove described formulation disposed within the squeezable container 72. A drop dispensing tip 80 enables drop wise 84 dispensing of formulation by squeezing of the container 72 as indicated by the arrows 86. In storage, the tip 80 is covered by a cap assembly 98.

With reference to both 5 and 6, the applicator device 70 includes a brush 100 securable for storage by a sheath 102 and removably affixed therein by external threads 104 in the cap 98.

As illustrated in FIG. 6 when the brush 100 is reversed within the sheath 102 with the brush 100 extending from the sheath 102 with the cap 98 secured by threads 105, the brush 100 and sheath 102 provide, in combination, a handle of sufficient length for facilitating the application of the formulation to the eyelid 32, as illustrated in FIGS. 1 and 2.

After application, the brush 102 and cap 98 are again reversed for insertion into the sheath 102 as illustrated in FIG. 5. This embodiment 70 provides close control over the amount of formulation applied to the brush 100 and thereafter to the eyelid 32 which, as hereinabove noted, should be between about 20 and 50 ml.

An aqueous solution containing 5%, by weight, bimatoprost is prepared as follows. Bimatoprost is dissolved in water and the resulting solution is sterilized by filtration. The solution is aseptically filled into the sterile container 14. Thereafter, as hereinbefore stated, the container is squeezed to prevent the solution to an eyelid and/or eyelash through the brush 24.

### EXAMPLE

Twenty-five healthy female volunteers completed the study
Ages: 30's and 60's
No history of glaucoma or other ocular diseases
No history of known allergy to Lumigan®
Strongly motivated by the possibility of longer, darker, and thicker natural eyelashes
Six clinic visits over a four month period of time (week 1, week 2, week 4, week 8, week 12, and week 16)
At each visit, the following procedures will be performed:
   Visual acuity
   Intraocular pressure (IOP)
   Slit lamp exam (SLE)
   Photography, front and side views
   Survey questionnaires during the course of the study
   No controls
One drop of Lumigan® was placed on the tip of an applicator
The medication was applied to the lid margin of the right upper eyelid just before bedtime
The process was repeated for the left upper eyelid
Patients were given a new sample bottle of Lumigan® at the beginning of the study and at the week 4 and at the week 8 visit
Patients discontinued using Lumigan® after the week 12 visit
Initially, 32 individuals requested to be in the study, but due to scheduling issues and other conflicts, only 29 individuals received the first dose of Lumigan®.
It took only 1 week to recruit these 32 interested participants
Early in the study, 4 of the 29 individuals who were given the medication and did not continue in the study scheduling issues and other conflicts
The study was completed with 25 individuals
Follow-up appointments were scheduled on Saturday mornings

### Preliminary Findings

Minimal Side Effects
   Vision
   IOP
   Ocular irritation
   Periocular pigmentary changes
Every participant grew longer lashes; many grew darker and thicker lashes as well as additional lashes
Nearly all of the participants are reluctant to stop using the Lumigan®
Patients grew lower lid lashes and eyebrow hairs too when they applied the Lumigan® “on their own” to these areas

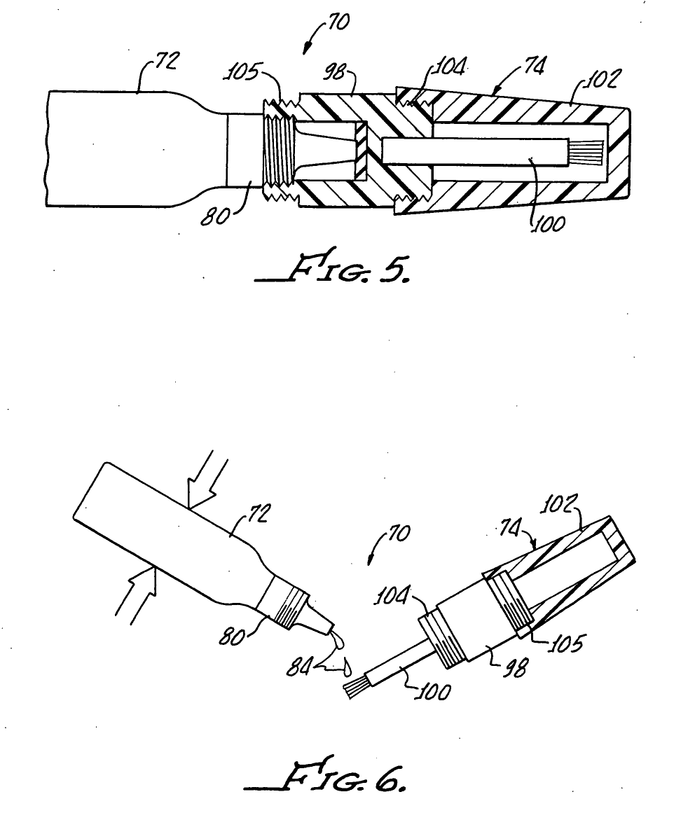

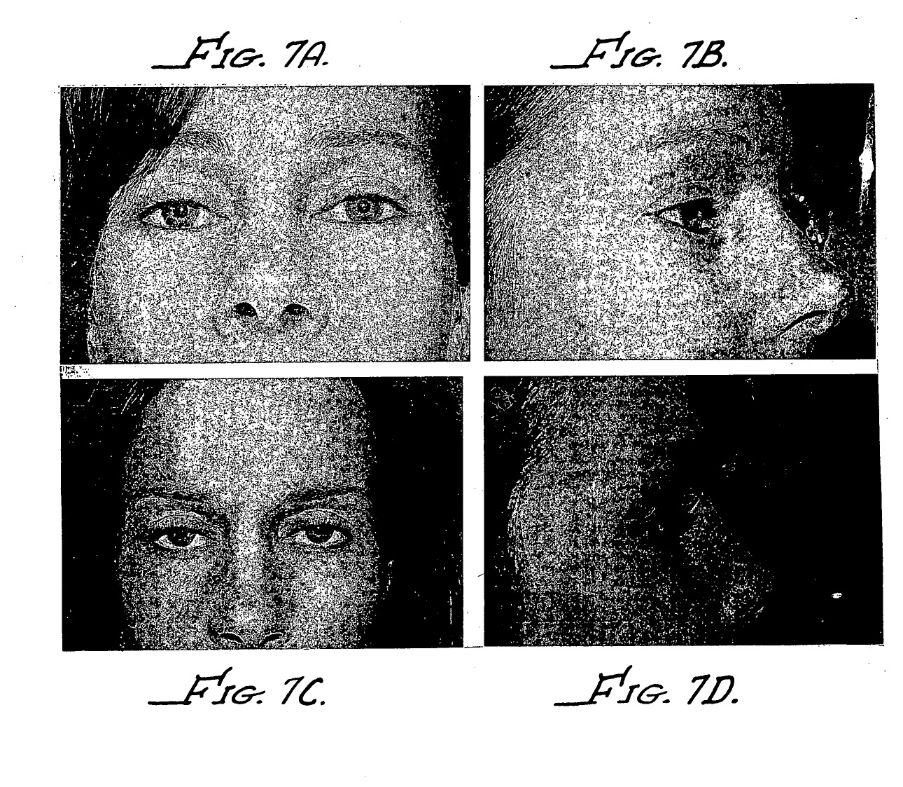

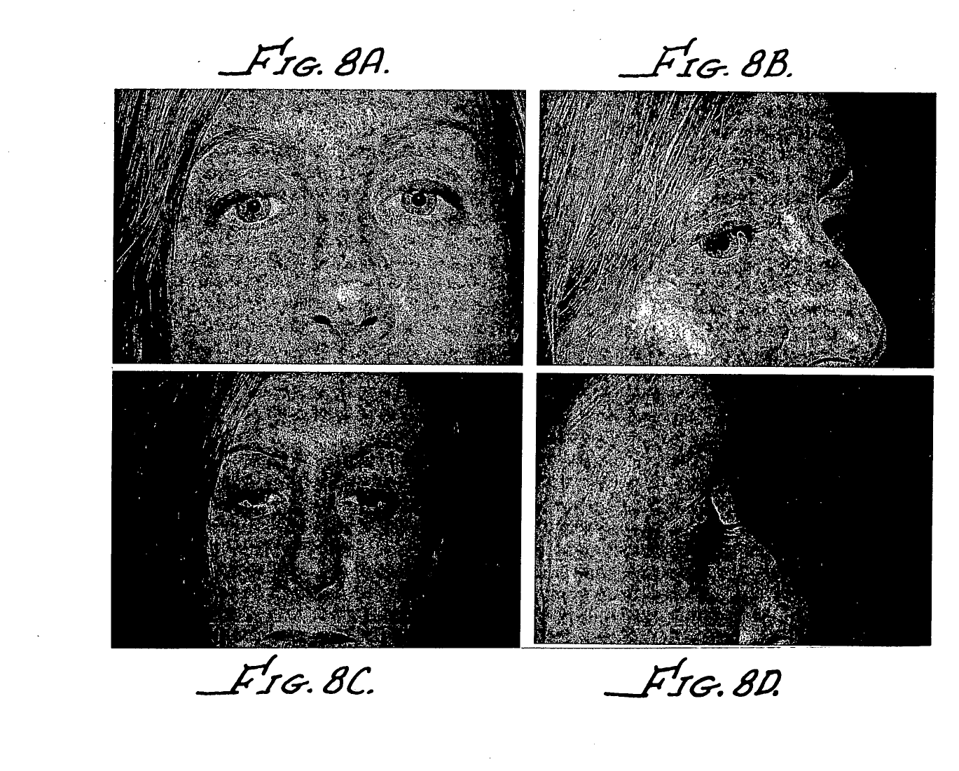

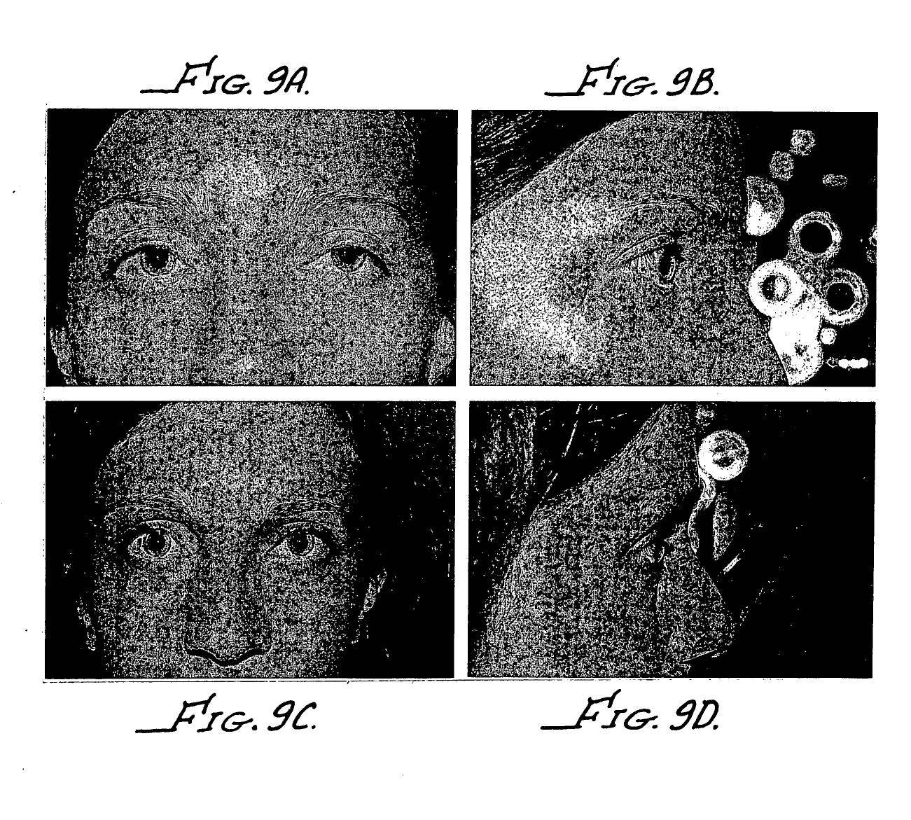

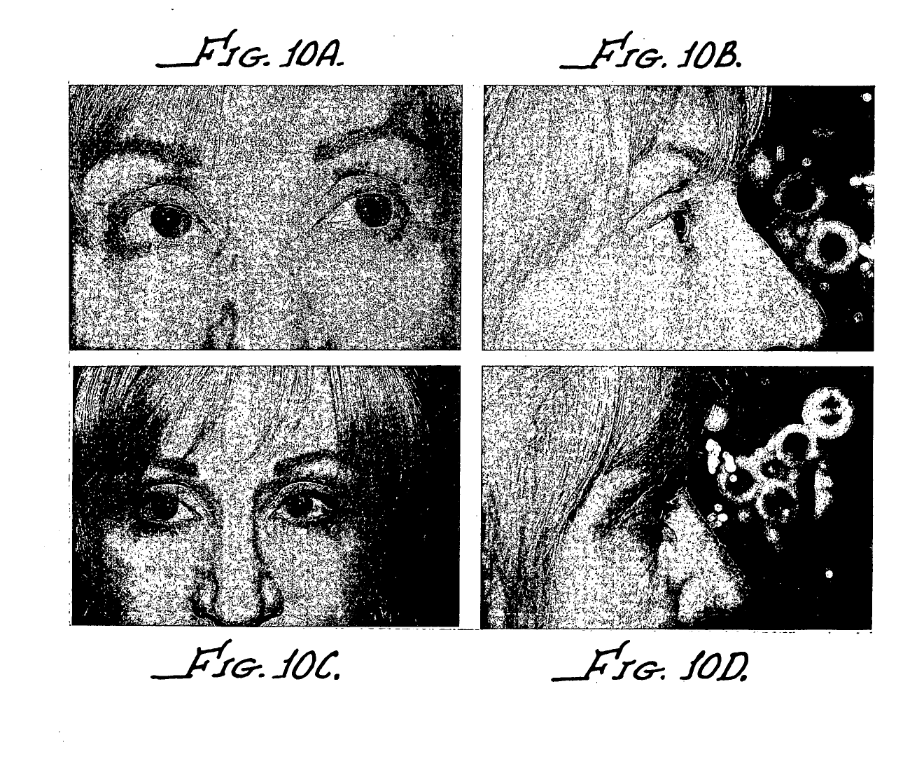

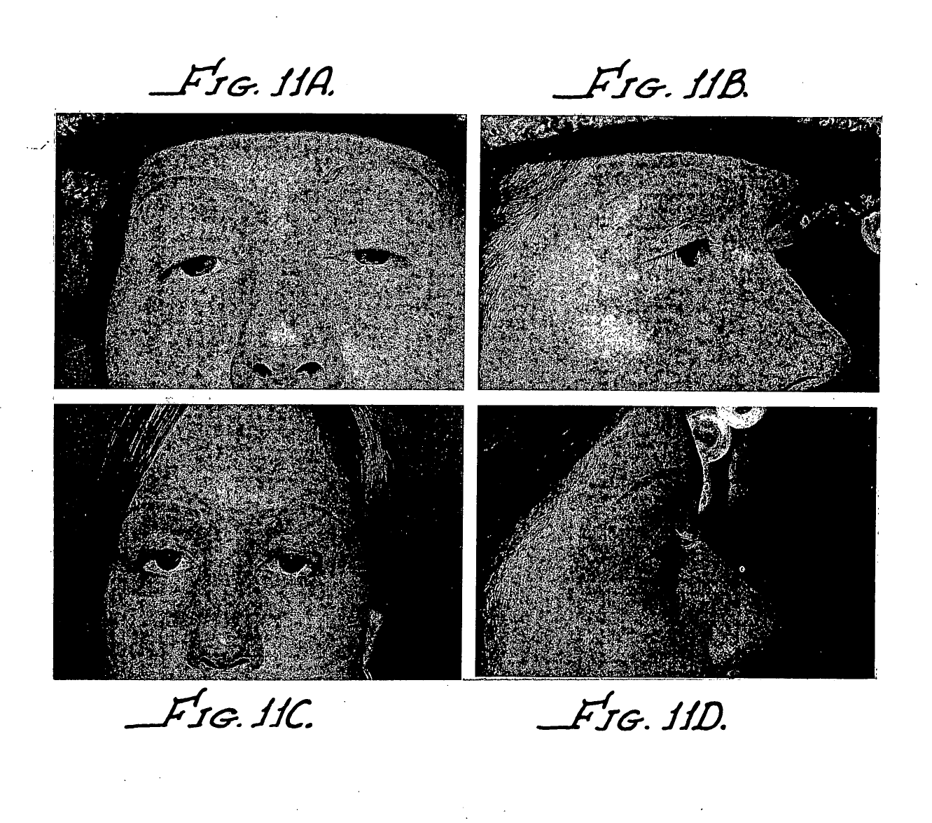

The results are shown in FIGS. 7A-7D, 8A-8D, 9A-9D, 10A-10D and 11A-11D. These substantiate that the participants grew longer lashes, many grew darker and thicker lashes as well as additional lashes.

Although there has been hereinabove described a specific eyelash applicator and method in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

What is claimed is:

1. A method of applying a hair growth stimulation formulation comprising:
applying to mammalian skin a volume of said formulation with an applicator device;
said formulation comprising a cyclopentane heptanoic acid, 2-cycloalkyl or arylalkyl compound represented by the formula I: 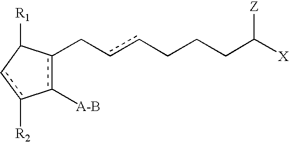
wherein dashed bonds represent a single or double bond which can be in a cis or trans configuration, A is an alkylene or alkenylene radical having from two to six carbon atoms, which radical may be interrupted by one or more oxa radicals and substituted with one or more hydroxy, oxo, alkyloxy or alkylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms, 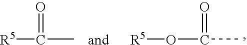
wherein R⁵ is a lower alkyl radical having from one to six carbon atoms; Z is ═O; one of R₁ and R₂ is ═O, —OH or a —O(CO)R₆ group, and the other one is —OH or —O(CO)R₆, or R₁ is ═O and R₂ is H, wherein R₆ is a saturated or unsaturated acyclic hydrocarbon group having from 1 to about 20 carbon atoms, or —(CH₂)mR₇ wherein m is 0 or an integer of from 1 to 10, and R₇ is cycloalkyl radical, having from three to seven carbon atoms, or a hydrocarbyl aryl or heteroaryl radical, as defined above or a pharmacologically acceptable acid addition salt thereof;
said formulation being packaged in a container preventing contamination of the formulation.

2. The method of claim 1 wherein the concentration of the compound applied is from about 0.0000001% to about 50% by weight of the composition.

3. The method of claim 1 wherein the compound is a compound of a formula III: 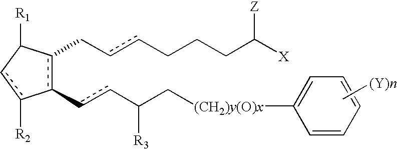
wherein y is 0 or 1, x is 0 or 1 and x and y are not both 1, Y is a radical selected from the group consisting of alkyl, halo, nitro, amino, thiol, hydroxy, alkyloxy, alkylcarboxy, halo substituted alkyl wherein said alkyl radical comprises from one to six carbon atoms, n is 0 or an integer of from 1 to about 3 and R₃ is ═O, —OH or —O(CO)R₆ wherein R₆, hatched lines indicate α configuration and solid triangles are used to indicate β configuration.

4. The method of claim 3 wherein the compound applied is bimatoprost in the form of a free base or acid addition salts thereof.

5. The method of claim 1 wherein a maximum of 3 ml of said formulation is disposed in said container.

6. The method of claim 1 wherein the applied volume of said formulation is between about 20 and about 50 microliters.

7. The method of claim 1, wherein said applicator device is selected from the group consisting of an applicator brush, porous foam swab or pad, hollow tube, eye dropper, dip stick, or combination thereof.

8. The method of claim 3 wherein the compound is bimatoprost or a pharmaceutically acceptable salt thereof.

9. The method of claim 7 wherein said applicator device is reusable.

10. The method of claim 7 wherein said volume of formulation is applied via a brush interconnected with said container.

11. The method of claim 10 further comprising covering said brush with a vented cap after application of the formulation.

12. The method of claim 10 wherein said volume of formulation is applied through a one-way valve disposed in said container.

13. The method of claim 10 wherein said volume of formulation is applied through a narrow neck interconnecting said container and said brush.

14. A method of applying eyelash growth stimulation formulation comprising:
applying to a mammalian eyelid a volume of said formulation with an applicator device;
said formulation comprising a cyclopentane heptanoic acid, 2-cycloalkyl or arylalkyl compound represented by the formula I: 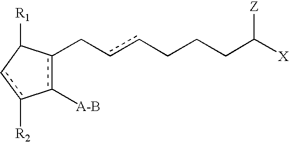
wherein dashed bonds represent a single or double bond which can be in a cis or trans configuration, A is an alkylene or alkenylene radical having from two to six carbon atoms, which radical may be interrupted by one or more oxa radicals and substituted with one or more hydroxy, oxo, alkyloxy or alkylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms, 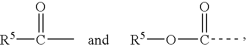
wherein R⁵ is a lower alkyl radical having from one to six carbon atoms; Z is ═O; one of R₁ and R₂ is ═O, —OH or a —O(CO)R₆ group, and the other one is —OH or —O(CO)R₆, or R₁ is ═O and R₂ is H, wherein R₆ is a saturated or unsaturated acyclic hydrocarbon group having from 1 to about 20 carbon atoms, or —(CH₂)mR₇ wherein m is 0 or an integer of from 1 to 10, and R₇ is cycloalkyl radical, having from three to seven carbon atoms, or a hydrocarbyl aryl or heteroaryl radical, as defined above or a pharmacologically acceptable acid addition salt thereof;
said formulation being packaged in a container preventing contamination of the formulation.

15. A hair growth application system comprising:
a squeezable container;
an applicator device; and
a formulation disposed in the container, said formulation comprising a cyclopentane heptanoic acid, 2-cycloalkyl or arylalkyl compound represented by the formula I: 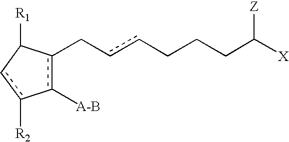
wherein dashed bonds represent a single or double bond which can be in a cis or trans configuration, A is an alkylene or alkenylene radical having from two to six carbon atoms, which radical may be interrupted by one or more oxa radicals and substituted with one or more hydroxy, oxo, alkyloxy or alkylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms, 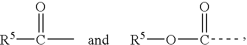
wherein R⁵ is a lower alkyl radical having from one to six carbon atoms; Z is ═O; one of R₁ and R₂ is ═O, —OH or a —O(CO)R₆ group, and the other one is —OH or —O(CO)R₆, or R₁ is ═O and R₂ is H, wherein R₆ is a saturated or unsaturated acyclic hydrocarbon group having from 1 to about 20 carbon atoms, or —(CH₂)mR₇ wherein m is 0 or an integer of from 1 to 10, and R₇ is cycloalkyl radical, having from three to seven carbon atoms, or a hydrocarbyl aryl or heteroaryl radical, as defined above or a pharmacologically acceptable acid addition salt thereof.

16. The system of claim 15 wherein the concentration of the compound is from about 0.0000001% to about 50% by weight of the composition.

17. The system of claim 15 wherein the compound is a compound of formula III: 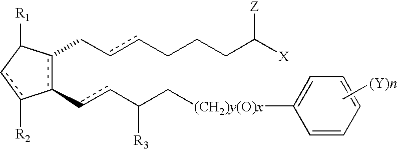
wherein y is 0 or 1, x is 0 or 1 and x and y are not both 1, Y is a radical selected from the group consisting of alkyl, halo, nitro, amino, thiol, hydroxy, alkyloxy, alkylcarboxy, halo substituted alkyl wherein said alkyl radical comprises from one to six carbon atoms, n is 0 or an integer of from 1 to about 3 and R₃ is ═O, —OH or —O(CO)R₆, hatched lines indicate α configuration and solid triangles are used to indicate β configuration.

18. The system of claim 17 wherein the compound applied is bimatoprost in the form of the free base or acid addition salts thereof.

19. The system of claim 15 wherein a maximum of 3 ml of said formulation is disposed in the container

20. The system of claim 15 wherein the compound is a compound of a formula. 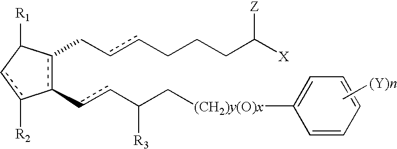
wherein y is 0 or 1, x is 0 or 1 and x and y are not both 1, Y is a radical selected from the group consisting of alkyl, halo, nitro, amino, thiol, hydroxy, alkyloxy, alkylcarboxy, halo substituted alkyl wherein said alkyl radical comprises from one to six carbon atoms, n is 0 or an integer of from 1 to about 3 and R₃ is ═O, —OH or —O(CO)R₆ wherein R₆, hatched lines indicate α configuration and solid triangles are used to indicate β configuration.

21. The system of claim 20 wherein the compound applied is bimatoprost in the form of a free base or acid addition salts thereof.

22. The system according to claim 15 wherein said applicator device is selected from the group consisting of an applicator brush, porous foam swab or pad, hollow tube, eye dropper, dip stick, or combination thereof.

23. The system according to claim 15 wherein said applicator device is a brush and the system further comprises a passageway interconnecting said brush and the container.

24. The system according to claim 23 further comprising a one-way valve disposed in one of the container and the passageway for preventing flow into the container.

25. The system according to claim 24 wherein said brush is sized for application of formulation to an eyelid.

26. The system according to claim 22 further comprises a breathable cap covering said applicator device and having vent holes therein.
